(19) United States
(12) Patent Application Publication
(10) Pub. No.: US 2025/0152877 A1
(43) Pub. Date: May 15, 2025

(54) APPARATUS FOR ADMINISTERING AN AEROSOL TO THE NASAL CAVITY, ACTIVATING DEVICE AND AEROSOL GENERATING DEVICE

(71) Applicant: SMART MEDICAL MUNICH UG, München (DE)

(72) Inventors: Florian MATL, Gauting (DE); Berthold POHL, Krailling (DE)

(21) Appl. No.: 18/833,799

(22) PCT Filed: Jan. 25, 2023
(86) PCT No.: PCT/EP2023/051818

§ 371 Date: Jul. 26, 2024

(30) Foreign Application Priority Data
Jan. 26, 2022 (DE) 10 2022 000 297.3

(51) Int. Cl. A61M 15/08; A61M 15/00

(52) U.S. Cl. CPC A61M 15/08; A61M 15/0098; A61M 2205/332; A61M 2205/3334; A61M 2205/3368; A61M 2205/3592; A61M 2205/58

(57) ABSTRACT

The present invention relates to an apparatus for administering an aerosol to the nasal cavity of a person, as well as to an activating device and an aerosol generating device of such an apparatus. Further described is a method for generating an aerosol for administration to the nasal cavity.

The present application relates to an apparatus for administering an aerosol to the nasal cavity of a person, as well as to an activating device and an aerosol generating device of such an apparatus. Further described is a method for generating an aerosol for administration to the nasal cavity.

An aerosol is a mixture of solid and/or liquid particles suspended in a gas. The aerosol to be administered to the nasal cavity may contain a substance such as a powdered and/or liquid medication, a topical medication, a cleaning agent, a rinsing solution and/or combinations thereof. The substance may be transferred into the aerosol by the device and then delivered by the device into a person's nose and thereby delivered to the person's nasal cavity.

Devices for administering substances to the nasal cavity are already known. The publication EP 2 340 865 B1 describes various nasal administration devices with an exhalation unit with a mouthpiece and a delivery unit with a nosepiece. The air exhaled into the mouthpiece can, for example, itself entrain a medication or can generate another air stream that entrains a medication, and this air stream carrying the medication can be delivered into the person's nose via a nosepiece. However, the administration device described in the aforementioned publication is not always reliable from a medical point of view, for example because the user can potentially influence the nasal administration in an undesirable way by unintentionally varying his exhalation into the mouthpiece. In addition, the delivery device is cumbersome to use and it is difficult to adapt the delivery device flexibly to different dosages, application requirements and aerosol generation mechanisms.

In view of the problems described, it is the object of the present invention to provide an apparatus for administering an aerosol to the nasal cavity of a person which is easy to handle, flexible in use and reliable from a medical point of view. Furthermore, an activation device and an aerosol generating device of such a device are provided.

This object is solved by an apparatus for administering an aerosol to the nasal cavity according to claim 1, an activating device (activation device) according to claim 6 and an aerosol generating device according to item 30. Advantageous further embodiments are described in the dependent claims.

The apparatus according to the invention comprises an aerosol generating device with an atomizer (nebulizer) for generating the aerosol and an activating device. The activating device comprises an blow-in opening, a first control unit adapted to activate the aerosol generating device by means of an activation signal in response to an exhalation of a person into the blow-in opening, and a blowing resistor adapted to resist the exhalation of the person.

In a preferred embodiment, the first control unit is set up for wireless activation of the aerosol generation device, in particular via radio and/or by means of an IR signal. For example, the first control unit comprises an electronic circuit, such as a microcontroller, which causes the activation signal to be sent to the aerosol generation device when a person exhales into the blow-in opening (injection opening), for example with a transmitter unit, in particular by means of a radio transmitter unit or an IR emitter. The activation signal may trigger aerosol generation by the atomizer (nebulizer) when received by the aerosol generation unit. In some embodiments, the first control unit may comprise an active or passive transponder and the activation signal is a transponder signal. In some embodiments, the first control unit may comprise a radio transmitter and the activation signal is transmittable in the form of an electromagnetic wave as a radio signal. The signal may be a useful signal modulated onto a carrier wave and transmitted via an antenna. In some embodiments, the activation signal can be an electrical signal that can be transmitted, for example via a cable—i.e. not necessarily wirelessly—from the activation device to the aerosol generation device. Optionally, communication between the activating device and the aerosol generation device can take place via a known standard such as Wi-Fi (WLAN) or Bluetooth directly or indirectly, e.g. via a router.

In some embodiments, which can be combined with other embodiments described herein, the activating device and/or the aerosol generation device are connected to a common network, such as a WLAN or LAN network, and communication takes place via the network. Communication via several networks, to which the activation device or the aerosol generation device can be connected directly or indirectly, is also conceivable. In particular, the activation signal is not necessarily transmitted directly from the activation device to the aerosol generation device, but optionally indirectly or indirectly, e.g. via a switch, a router or a gateway. In this case, the activation signal can be the “useful signal” for activating the aerosol generation device, which, with regard to wireless transmission, can be transported by means of one or more carrier signals on any transmission path via one or more intermediate stations until it is received by the aerosol generation device. The first control unit can have all the necessary means for communication in one or more networks or for direct communication, e.g. by means of Bluetooth.

The exhalation of a person into the injection opening can be conveniently detected by means of a sensor provided in the activation device, such as a pressure sensor or flow sensor, whereupon the first control unit can initiate the activation of the aerosol generation by transmitting the activation signal.

In a preferred embodiment, the aerosol generation device has a second control unit that is set up to trigger aerosol generation by the atomizer when the activation signal is received. For example, the second control unit has a receiver for receiving the activation signal from the activating device, such as a radio receiver or IR receiver. In some embodiments, the second control unit comprises an electronic circuit, such as a microcontroller, which is arranged to trigger aerosol generation by the atomizer upon receipt of the activation signal. The activating device and the aerosol generation device can be connected to each other wirelessly, in particular via radio, or alternatively via a cable. If a cable is provided, the activation signal can be transmitted to the aerosol generation device, for example in the form of an electrical signal. The second control unit is preferably, but not necessarily, present in the aerosol generation device. For example, the activation signal can be transmitted directly to the atomizer (nebulizer) to trigger aerosol generation, e.g. via a cable.

According to the embodiments described herein, the first control unit can be used to activate the aerosol generating device extremely reliably in response to the person exhaling into the injection opening. Furthermore, the activation signal can optionally not only trigger the activation of the aerosol generation, but also transmit further information for nasal administration to the aerosol generation device, e.g. information regarding dosage, aerosol temperature, aerosol flow rate, aerosol delivery time or the like. This enables flexible use of the apparatus and adaptation to different applications.

According to embodiments described herein, the activating device acts as a wired or wireless “remote control” for the aerosol generating device, and the aerosol generating is activated by means of the activation signal. This enables convenient handling of the device because exhalation can be performed by the user into the blowing opening of the activation device, while the aerosol generating device can be held separately from the activation device at the user's nose to administer the aerosol to the nasal cavity. Despite the fact that the aerosol generation device and the activation device can be handled separately, the aerosol generation can always be activated reliably by means of the activation signal.

An activating device (activation device) of an apparatus for administering an aerosol to the nasal cavity is also separately described and claimed herein. Such an activating device comprises a blow-in opening (injection port) into which a person can exhale, a first control unit adapted to transmit an activation signal to an aerosol generating device in response to a person exhaling into the blow-in opening, and a blowing resistor adapted to resist the person exhaling into the blow-in opening. The activating device may further comprise any features of the activating device of the apparatus described herein and hereinafter, in particular a sensor, a display, a mouthpiece, a first connecting portion, a receiving portion for receiving a first energy storage device and/or a holding portion, etc.

Furthermore, a aerosol generating device of a device for administering an aerosol to the nasal cavity is also separately described and claimed herein. The aerosol generating device comprises a container for holding a substance, in particular a medicament, an atomizer (nebulizer) for converting the substance into an aerosol and a dispensing opening through which the aerosol can be administered to the nasal cavity of a person. The aerosol generating device further comprises a second control unit which is arranged to trigger aerosol generation by the atomizer (nebulizer) upon receipt of an activation signal from an activating device, in particular from the activating device described herein. The aerosol generating device may further comprise any features of the aerosol generating device of the apparatus described herein and hereinafter, in particular a receiving portion for receiving a second energy storage device, a dispensing port with nose engaging portion, a second connecting portion and/or a display, etc.

According to some embodiments, which may be combined with other embodiments, the activating device and the aerosol generating device are not attached to each other when in use. In particular, the activating device and the aerosol generating device may be physically separated from each other. For example, the activating device and the aerosol generating device can be held with two different hands to use the apparatus and can be brought to the mouth or nose separately from each other, which simplifies handling of the apparatus. In alternative embodiments, the activating device and the aerosol generating device can also be connected to each other, e.g. attached to each other, during use, i.e. during exhalation into the blow-in opening.

Optionally, the activating device and the aerosol generating device can be detachably attached to each other, for example by means of corresponding connecting means provided on the activating device and/or the aerosol generating device. The device can then be held or stowed in “one-piece form”, for example in the idle state, when the activating device and the aerosol generating device are attached to each other. This can further improve the manageability of the device.

Preferably, for this purpose, the activating device has a first connecting part for detachable attachment to the aerosol generating device. Alternatively or additionally, for this purpose, the aerosol generating device has a second connecting part for detachable attachment to the activating device. The first connecting part can have at least one magnetic element which is set up for coupling to at least one second magnetic element of the aerosol generating device. The first and/or the second magnetic element may be ferromagnetic. At least one of the magnetic elements can be a permanent magnet. In this way, the activating device and the aerosol generating device can be attached to and detached from each other quickly and easily.

According to a particularly preferred embodiment, the activating device is adapted to close the soft palate (the oropharyngeal velum) of the person exhaling into the injection port. In other words, the activating device causes the closure of the soft palate of a person when that person exhales into the blow-in opening.

In particular, the nasal cavity comprises the nasal airway with the nasal cavities separated by the nasal septum and numerous ostia, e.g. paranasal sinuses and tubal ostia, and is covered with the nasal mucosa. The nasal cavity can also include the nasopharynx, the Eustachian tubes and the tympanic cavities. The nasal airway communicates with the nasopharynx, the oral cavity and the lower airway, whereby the soft palate is essentially located at the transition between the nasal airway and the oral cavity or at the transition between the nasopharynx and the oral cavity. In an open position, the soft palate opens a connection between the nasal cavity and the oral cavity and closes this connection in a closed position. The closed position can be brought about by creating a predetermined excess pressure in the oral cavity in relation to the nasal cavity, as can occur, for example, when exhaling through the oral cavity. An exhalation of the person into the activation device, in particular against the blowing resistor, can therefore lead to the closure of the soft palate according to the embodiments described herein.

In particular, the soft palate is closed indirectly by generating an overpressure in the oral cavity relative to the nasal cavity by exhaling into the activating device. The closure of the soft palate by exhalation preferably takes place simultaneously or shortly before the aerosol generation is triggered by the activation signal, i.e. before the aerosol is administered to the nasal cavity, by building up pressure in the oral cavity. This ensures that most or all of the aerosol, which may contain a medication for local administration to the nasal cavity, is actually administered to the nasal cavity and does not or hardly penetrates into the oral cavity.

In preferred embodiments, the blowing resistor is set up to generate the intra-oral overpressure in the person exhaling into the blow-in opening, which leads to the closure of the soft palate. For example, the activating device comprises a cavity adjoining the blow-in opening, for example in the form of a tube section, into which the person can exhale, whereby the blowing resistor can be arranged in the tube section. The blowing resistor can act as a flow resistance and provide resistance to the person when exhaling, so that the person exhales against the blowing resistor, thereby increasing the person's intra-oral pressure when exhaling into the activating device. The blowing resistor can, for example, be formed as a constriction, a flap, a membrane, an elastic balloon, a baffle plate, a deflector plate or the like in a tube section that adjoins the blow-in opening. For example, the blowing resistor can have a plurality of deflector plates in a pipe section. The blowing resistor can be arranged inside a cavity formed in the activation device. The blowing resistor can be stationary (e.g. as a constriction of a pipe section) or movable (e.g. as a flap pretensioned into a closed position). The blowing resistor can be adjustable to enable control of the intra-oral pressure generated during exhalation.

According to some embodiments, the activating device has a mouthpiece forming the blow-in opening, through which the person can exhale into the blow-in opening. The mouthpiece can be detachably or firmly connected to a base body of the activating device. For example, the mouthpiece can be replaced or cleaned after repeated use and can be removed for this purpose. The mouthpiece can be designed as a tubular section that enables blowing through. The blowing resistor can be arranged inside the mouthpiece.

Preferably, the mouthpiece is arranged on the base body of the activation device so that it can rotate about a longitudinal axis L. The activating device has a release element, such as a knob or a button, which can be actuated to release a rotation of the mouthpiece relative to the base body. The mouthpiece can be rotated by a predetermined angle, such as 90° relative to the base body, in order to activate or “switch on” the activation device, so that the activating device is ready to transmit the activation signal when an exhalation is detected. This can lead to improved handling of the activating device. Turning the mouthpiece back to the starting position can deactivate or “switch off” the activating device again. Other switch-on or switch-off mechanisms are also conceivable.

The activating device can also have a sensor for detecting an exhalation into the blow-in opening, such as a pressure sensor or a flow sensor. In particular, the activating device has a pressure sensor which is set up to detect a pressure or a change in pressure in the activating device, for example inside the mouthpiece or the pipe section formed by it. The pressure sensor can be arranged inside a cavity formed in the activating device upstream of the blowing resistor. A pressure measured by the pressure sensor or a change in pressure can be transmitted to the first control unit. If the pressure or the pressure change exceeds a predetermined value (so that, for example, closure of the soft palate is reliably ensured), the first control unit can initiate or execute transmission of the activation signal to the aerosol generating device.

The activating device can also have a display that is set up to indicate an operating state of the activating device. For example, the display is activated when a predetermined pressure is reached when the person exhales into the blow-in opening, which triggers the generation of aerosol. The indicator can show visually, acoustically and/or tactilely when the predetermined pressure is reached. For example, the indicator can have a light indicator such as an LED, an acoustic indicator that emits a sound or another audio signal and/or a vibration generator.

Alternatively or additionally, the aerosol generating device may have a display, which is set up to display, an operating state of the aerosol generating device, for example a visual, acoustic and/or tactile display. For example, the indicator may be activated during the administration of the aerosol and/or it may be deactivated when no aerosol is being administered.

Conveniently, the activating device can be set up to hold a first energy storage device, in particular a battery, to provide energy for the activation device. For example, the activating device has a battery compartment for holding one or more batteries such as button cells. The first energy storage device can provide the energy for operating the first control unit, the pressure sensor, the display and/or for transmitting the activation signal, for example via a radio transmitter unit. In some embodiments, however, a first energy storage unit is not provided, for example if the first control unit has a passive transponder which can be powered, for example, by a field emanating from the aerosol generating device. A first energy storage device is also not necessarily required if the activating device and the aerosol generating device are connected via a cable, whereby electrical energy can also be transmitted via the cable in addition to the activation signal.

Alternatively or additionally, the aerosol generating device is set up to accommodate a second energy storage device, in particular a battery, to provide energy for aerosol generation and/or its triggering. For example, the aerosol generating device has a battery compartment for holding one or more batteries such as button cells. The second energy storage device can provide the energy for operating the atomizer, the display, the second control unit, for receiving the activation signal and/or for triggering the aerosol generation. In some embodiments, however, a second energy storage device is not provided, for example if the atomizer has a pressurized gas as an energy source and/or if an energy required for the operation of the aerosol generating device can be provided wirelessly or by cable via the activation device.

The manageability of the activating device can be improved if the activating device has at least one holding area for gripping and holding the activating device with a hand or a part thereof during use. The at least one holding area can have at least one recess that can be adapted to the engagement of a finger, so that the activating device can be held comfortably with one hand. For example, two holding recesses pointing in opposite directions can be provided on a base body of the activating device for engagement of the thumb and index finger.

The aerosol generating device preferably has, in addition to the atomizer (nebulizer), a container set up to hold a substance, the nebulizer being set up to transfer the substance into the aerosol. The substance may comprise one or more of the following components: one or more drugs, a cleaning agent for cleaning the nasal cavity, a drug-containing solid such as a powder or a drug-containing liquid. In the treatment of nasal diseases, local (topical) administration of medication to the nasal cavity may be preferable (e.g. cortisone such as mometasone furoate). Alternatively or additionally, however, such drug administration to the nasal cavity may also be preferable in order to achieve a systemic effect, e.g. for the prophylaxis and/or treatment of diseases such as osteoporosis, migraine, as hormone replacement therapy (e.g. insulin administration), or as a vaccination. The container can be adapted to the substance to be administered, e.g. to the intake of a powder and/or a liquid and/or to a quantity of the substance to be dispensed for one or more applications.

In addition to the atomizer, the aerosol generating device also preferably has a dispensing opening through which the aerosol can be administered to the nasal cavity of the person. Preferably, the aerosol generating device has a nose engagement piece forming the dispensing opening, which can optionally be arranged on the aerosol generating device so as to be rotatable about an axis of rotation. The nose engagement piece can be removably attached to the aerosol generating device so that it can be removed for cleaning or replacement after repeated use. This also allows the use of a nosepiece adapted to a specific nose size. The nose engagement piece can, for example, be detachably attached to a protruding end of an aerosol dispensing channel via a snap connection.

The nose engagement piece can be designed to engage in a nostril of the user and have a through opening through which the aerosol can flow into the nostril. The nasal engagement piece can be designed to achieve a liquid-tight seal towards the nostril. For example, the nasal engagement piece can be made of an elastic material such as an elastic polymer or silicone. The nose engagement piece can have a circumferential sloping surface so that it can be brought into sealing engagement with nostrils of different sizes. Optionally, two nose engagement pieces can also be provided, which form two discharge openings for simultaneous discharge of the aerosol into both nostrils.

Preferably, the nasal engagement piece is adapted to protrude more than 1 mm and less than 2 cm into a nostril when engaged with the nostril for administration of the aerosol. The shape of the nasal engagement piece may be adapted to specific needs. For example, the nasal engagement piece may be shaped to promote turbulence formation and/or good dispersion of the aerosol. In particular, the nose engagement piece may have a nose olive that can contribute to the formation of a suitable physiological gas flow.

The aerosol generating device may comprise a sensor adapted to determine a position and/or location of the aerosol generating device, in particular with respect to the nose or nasal opening of the user. For example, the sensor may be adapted to measure a position and/or location of the nose engagement piece of the aerosol generating device, in particular with respect to the nose or nasal opening of the user into which the nose piece projects for aerosol delivery.

In some embodiments, a display can be provided which is set up (configured) to generate an (e.g. acoustic optical and/or tactile) display signal as a function of a measurement signal from the sensor. For example, the display can be set up (configured) to generate a (first) display signal when the aerosol generation device or its nose engagement piece assumes a predetermined position and/or orientation, in particular in relation to the user's nose or nose opening. The user can recognize by the (first) display signal that he is currently holding the aerosol generating device in a correct position and can then trigger the aerosol generation by exhaling into the blow-in opening. Alternatively or additionally, the display can be set up to generate a (second) display signal if the aerosol generating device or its nose engagement piece is not arranged in a predetermined position and/or orientation. The user can use the (second) display signal to recognize that he is not holding the aerosol generating device in a correct position and can correct its position, for example by moving, tilting or repositioning the nose engagement piece in the nose opening, before triggering aerosol generation.

Alternatively or additionally, a control unit of the aerosol generating device (in particular the second control unit) can be set up in such a way that it triggers the aerosol generation by the atomizer or the administration of the aerosol to the nasal cavity as a function of a measurement signal from the sensor. For example, the control unit is set up in such a way that it only triggers the aerosol generation or the administration of the aerosol when the aerosol generation device or its nasal engagement piece assumes a predetermined position and/or orientation, in particular in relation to the nose or the nasal opening of the user. For this purpose, a measurement signal from the sensor can be transmitted to the control unit so that the control unit can trigger the aerosol generation as a function of the sensor signal. In this way, misdirection of the aerosol can be prevented in cases where the user holds the aerosol generating device in an incorrect position and/or orientation against his nose. In particular, the probability of a misdirected (e.g. past the nose) or reduced (e.g. due to a poor flow angle) aerosol flow can be reduced.

The sensor can, for example, have a distance or range sensor, such as a laser or LED range finder or a non-optical or optical proximity switch such as an IR distance sensor. The sensor can determine a free path length from the aerosol generating device. In some embodiments, the sensor may be located in the region of the nasal engagement piece, for example at or in the aerosol dispensing opening of the device, preferably such that it “shines” straight into the nasal cavity when the aerosol generating device is correctly positioned. Correct positioning can be recognized by a measured value of the sensor within a predetermined value range. For example, a measured value of the sensor below a lower threshold value can indicate an incorrect insertion angle of the nasal engagement piece into the nasal opening, and/or a measured value of the sensor above an upper threshold value can indicate that the nasal engagement piece does not yet protrude into the nasal opening at all or is completely incorrectly positioned.

Alternatively or additionally, the aerosol generation direction can comprise a position sensor (e.g. a magnetometer or gyroscope) for determining a position or orientation of the aerosol generation device. The measured value of the position sensor can be used, for example, to detect whether the aerosol generation device is in a correct position (e.g. at an advantageous angle relative to the vertical with regard to aerosol release, which is in the range of the angle of typical nasal cavities relative to the vertical, etc.) and/or in a completely incorrect position (e.g. horizontal instead of vertical, etc.).

Other types of sensors for position and/or position determination are also conceivable, such as a camera, a pressure sensor on the nose insert or similar.

In some embodiments, which may be combined with other embodiments described herein, the aerosol generating device comprises a first sub-unit and a second sub-unit detachably connected to the first sub-unit. The first sub-unit has the atomizer, the container, the dispensing opening (or an aerosol dispensing channel with the dispensing opening), and the second sub-unit has the second control unit and/or a battery storage compartment. The first sub-unit can be removed from the second sub-unit, e.g. to change batteries or to refill a substance in the container. The first sub-unit can be connected to the second sub-unit via connecting elements. For example, the first sub-unit can be magnetically coupled to the second sub-unit. Each of the sub-units can have a housing section. Alternatively, the aerosol generating device can also have a one-piece housing that does not allow the removal of a separable sub-unit.

In some embodiments, which may be combined with other embodiments described herein, the aerosol dispensing channel may be arranged rotatable about a longitudinal axis L from the rest (i.e. from the base body) of the first sub-unit. Expediently, the first sub-unit comprises a release element, such as a knob or button, which is operable to release rotation of the aerosol dispensing channel relative to the base body. The aerosol dispensing channel can be rotated by a predetermined angle, such as 90° or 180°, relative to the base body. This can lead to improved handling of the aerosol generation device, as accessibility to the nose is facilitated.

In a preferred embodiment, the dispensing opening is arranged adjacent to the activation device when the activation device and the aerosol generating device are attached to each other. For example, the aerosol dispensing channel having the dispensing opening protrudes from the atomizer in a direction in which the activation device is attached to the aerosol generating device. In this way, a compact and handy device can be provided.

Preferably, the aerosol generating device has an aerosol delivery controller and/or an airflow accelerator for providing a predetermined aerosol flow rate and/or aerosol flow velocity that is delivered through the delivery port. The aerosol delivery controller can optionally be set up to maintain the predetermined aerosol flow rate over a predetermined period of time. The airflow accelerator, e.g. a fan or a micropump, can provide an inflow of aerosol into the nasal cavity at a predetermined speed and/or a predetermined rate. The aerosol delivery control can ensure the amount of aerosol to be generated by the nebulizer per unit of time or over a predetermined period of time, e.g. an aerosol flow rate and/or a total amount of aerosol to be delivered. Optionally, the activation signal can contain aerosol delivery information that influences the aerosol delivery, e.g. by setting the aerosol delivery control and/or the airflow accelerator accordingly.

In some embodiments, which may be combined with other embodiments described herein, the airflow accelerator is a micropump. Exemplary micropumps may be peristaltic systems. Peristaltic systems are an imitation of nature. Miniaturization offers great advantages, especially for non-mechanical pumps. Very high electric or magnetic field strengths can be achieved due to the small geometries. The effect-electrokinetic, electrohydrodynamic, electrohydromagnetic, electrochemical, etc.—is many times more effective than in the macro world. Preferably, the micropump used herein can contain a piezomembrane.

In some embodiments, which may be combined with other embodiments described herein, the airflow accelerator is a fan. Exemplary fans may be axial fans, diagonal fans, radial/centrifugal fans, or tangential or cross-flow fans.

In a preferred embodiment, the aerosol generating device is adapted to deliver an aerosol flow rate of 5 l/min or more and 50 l/min or less to the nasal cavity, preferably 10 l/min or more and 30 l/min or less, in particular about 15 l/min.

The aerosol generating device can have at least one heating element for heating the aerosol to a predetermined temperature. This enables the aerosol to be delivered to the nasal cavity at a predetermined delivery temperature.

The aerosol generating device may have a delivery device, such as a micropump, which is designed to deliver a predetermined amount of the substance to the atomizer when the aerosol generation is triggered.

The atomizer is designed to transfer a substance into an aerosol. “Atomizing” is broadly understood herein as transferring a substance (fluid or solid) into an aerosol and is not limited to spraying a liquid. Among other things, “atomization” also includes vaporization, nebulization, sublimation and spraying of powder. The transfer of the substance into the aerosol can take place in particular by one or more of the following mechanisms (1)-(6):

(1) By heating the substance with a heating element: The atomizer can have a heating element such as one or more heating coils or heating spirals (e.g. like a coil in an e-cigarette) for vaporizing the substance. A predetermined dosage of the substance can be brought into the vicinity of the heating element or into contact with the heating element for vaporization, e.g. by means of a conveying device such as a micropump or a wick. The generated vapor can be delivered to the nasal cavity as an aerosol, e.g. by diffusion through the dispensing opening or additionally by means of an airflow accelerator, such as a micropump or a fan. In particular, the atomizer can be set up in the manner of a vaporizer head of an e-cigarette.

(2) By causing the substance to vibrate via an ultrasonic element: The atomizer can have an ultrasonic element such as an ultrasonic nebulizer, which can have a piezoelectric element that can be caused to vibrate, for example. A predetermined dosage of the substance can be nebulized by causing it to vibrate, for example by applying it to a platelet of the ultrasonic element that can be caused to vibrate, in particular by means of a delivery element such as a micropump. The generated vapor can be delivered to the nasal cavity as an aerosol, e.g. by diffusion through the output opening or additionally by means of an airflow accelerator. In particular, the nebulizer can be set up in the manner of an ultrasonic nebulizer of an e-cigarette.

(3) The atomzer may have a device for passing a compressed gas through the substance. The substance can be entrained by the gas stream and the gas stream carrying the substance can be delivered to the nasal cavity as an aerosol. For example, the atomizer has a pressurized gas source for entraining a measured dosage of the substance, e.g. a powder containing medication.

(4) By means of a vibrating membrane: The atomizer can have a membrane with openings (e.g. micro-openings) that can be made to vibrate. A liquid substance arranged on one side of the membrane wets the membrane, passes through the openings and leaves the membrane on the other side as an aerosol. The vibrating membrane can be made to vibrate by means of a piezoelectric element, for example.

(5) The atomizer may have a nozzle through which the substance is sprayed to generate the aerosol, in particular using a propellant under pressure.

(6) The atomizer can have an inhalation device, such as a high-pressure inhaler with a predetermined dosage of the substance. The inhaler can be designed to vaporize the substance to provide the aerosol.

The atomization mechanism is not limited to the above examples, but includes any mechanism that is suitable for transferring the substance into the aerosol.

The aerosol generating device preferably has a second control unit for triggering aerosol generation by the atomizer on receipt of the activation signal from the activation device. The activation signal can trigger the generation of a predetermined quantity of aerosol and/or the generation of the aerosol over a predetermined period of time. Alternatively or additionally, the activation signal can be transmitted as long as the person exhales into the injection opening of the activation device, so that aerosol generation can be stopped as soon as the person no longer exhales and/or when a predetermined maximum amount of aerosol has already been delivered to the nasal cavity. Alternatively, a deactivation signal can be transmitted by the activation device as soon as the person no longer exhales into the injection opening, whereupon aerosol generation can be stopped.

An apparatus described herein can be used to generate an aerosol and to administer the aerosol to the nasal cavity of a person as long as the person exhales into an injection port of the activation device of the device. Exhalation into the blow-in opening results in the transmission of an activation signal from the activating device to the aerosol generating device, which triggers aerosol generation.

Further described herein is a method of generating an aerosol. The method comprises exhaling a person into blow-in opening of an activating device of an aerosol delivery apparatus. The method further comprises transmitting an activation signal from the activating device to an aerosol generating device of the apparatus in response to the exhalation.

The activation signal leads to aerosol generation by an atomizer of the aerosol generation device and to the administration of the aerosol to the nasal cavity of the person, in particular via a nasal engagement piece engaging in the person's nose.

In particular, the apparatus is an apparatus according to an embodiment described herein.

Preferably, the aerosol generating device and the activating device are not attached to each other when the person exhales into the blow-in opening.

Preferably, the activation signal is transmitted wirelessly, e.g. via radio, infrared radiation and/or by means of a transponder. Alternatively, a wired transmission can also be provided.

Preferably, the aerosol contains a medication.

In the following description, the invention is explained with reference to the accompanying drawings. It shows:

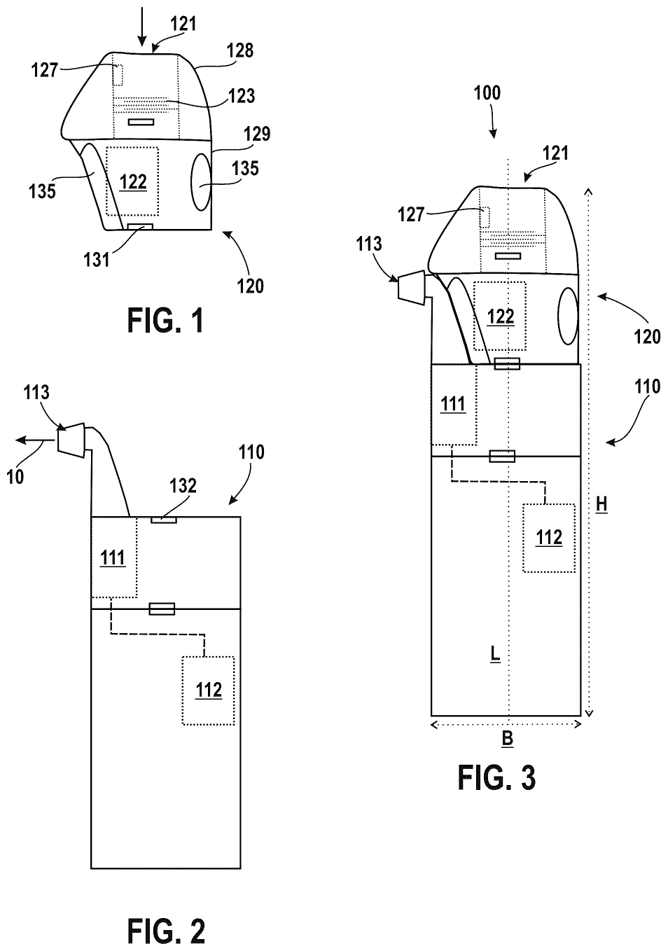

FIG. 1 an activating device according to the invention;

FIG. 2 an aerosol generating device according to the invention;

FIG. 3 an apparatus according to the invention in a coupled state; and

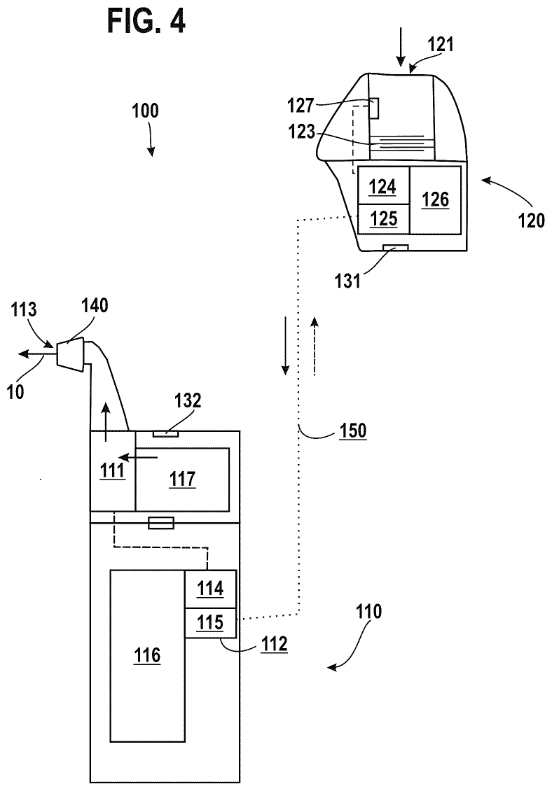

FIG. 4 an apparatus according to the invention when used in a separated state.

FIG. 1 is a schematic representation of an activating device 120 according to the invention, which represents a first portion of an apparatus 100 for administering an aerosol 10 to the nasal cavity. FIG. 2 is a schematic representation of an aerosol generating device 110 according to the invention, which represents a second part of the device 100. The entire apparatus 100 is shown schematically in a coupled state in FIG. 3, in which the activating device 120 and the aerosol generating device 110 are attached to each other.

As shown in FIG. 1, the activating device 120 includes an blow-in opening 121 into which a person can exhale and which is formed by a mouthpiece 128. The mouthpiece 128 may have a tubular portion (shown as a dashed line in FIG. 1) that allows the person to exhale through the mouthpiece 128.

The activating device 120 further comprises a blowing resistor 123, exemplified in FIG. 1 as a plurality of deflector plates. The blowing resistor 123 may be disposed within the tubular portion formed by the mouthpiece 128. The blowing resistor 123 is arranged to provide resistance to the person's exhalation, so that when the person exhales, a positive intra-oral pressure is established in the person's oral cavity, which leads to closure of the soft palate.

The mouthpiece 128 can be rotatably attached to a base body 129 of the activating device 120. The rotatability can be released by actuating a release element, for example by pressing a release button. A rotation of the mouthpiece 128 relative to the base body 129 by a predetermined angle (e.g. by) 90° can activate or switch on the activation device. Turning the mouthpiece 128 back to the initial position can deactivate or switch off the activating device.

The activating device 120 further comprises a first control unit 122 adapted to activate the aerosol generating device 110 in response to the person exhaling into the blow-in 121. The first control unit 122 may be adapted to transmit an activation signal to the aerosol generating device when a predetermined pressure or flow of breath is detected in the mouthpiece 128, for example by means of a sensor such as a pressure sensor 127 or a flow sensor.

The first control unit 122 may be arranged for wireless or wired activation of the aerosol generation unit 110, for example via cable or by means of a radio or IR signal. In particular, the first control unit 122 comprises an electrical circuit, such as an integrated circuit (IC), control chip or microcontroller, which is adapted to transmit the activation signal to the aerosol generating device 110 when an exhalation into the activating device 120 is detected. The activation signal may be sent wirelessly, for example by means of a transmitter with an antenna or an IR diode, which may be part of the first control unit 122.

As shown in FIG. 4, the first control unit may include an integrated circuit 124 having a transmitter 125 for wirelessly transmitting the activation signal 150. Optionally, the first control unit may comprise a receiver or a transceiver such that bidirectional communication with the aerosol generation device 110 is enabled. In one possible embodiment, the first control unit comprises a transponder with a microcontroller, for example a passive or active RFID transponder.

The activation signal 150 can be transmitted directly or indirectly (e.g. via WLAN) to a second control unit 112 of the aerosol generating device 110, which can have, for example, an integrated circuit 114 with receiver 115 (see FIG. 4), e.g. a radio receiver or a photodiode. Optionally, a transmitter or a transceiver may be provided so that bidirectional communication with the activating device 120 is enabled. Alternatively, the activation signal 150 can be transmitted directly to the atomizer 111 to trigger aerosol generation, for example via a cable or other electrical connection.

Optionally, the first control unit and/or the second control unit can have a control chip with a processor (e.g. with a CPU) and/or a data memory, e.g. a flash memory. Optionally, the first control unit and/or the second control unit is set up to store information relating to the aerosol administration on a data memory of the device, such as information on the duration, frequency and/or quantity of the aerosol administration, and/or to store information on the fill level of the container and/or the charge level of a battery. Optionally, the first control unit and/or the second control unit can be set up for communication via a network, e.g. via WLAN, and/or for (direct or indirect) communication with an external data processing device (such as a smartphone) via one or more known communication standards, such as Bluetooth or WLAN. Optionally, the device is set up for communication with an external data processing device, such as a mobile device (e.g. a smartphone), e.g. via Bluetooth or WLAN. For example, an external application can query information on an external data processing device that is stored in a data memory of the device according to the invention. In particular, the device according to the invention can be set up to interact with an external application that can be installed on a smartphone, for example. The communication can be bidirectional. For example, the device/apparatus can initiate a push message to an external application on a smartphone or transmit it itself, e.g. when an aerosol administration is pending. For example, a user can request information via an external application on a smartphone, e.g. when or how often an aerosol has already been administered to the nasal cavity, or what the current fill level of the container is. The smartphone can then request the corresponding information from the device using wireless communication, and the device/apparatus can transmit the information to the smartphone using wireless communication, for example if it is stored in a data memory of the device. Optionally, the device/apparatus can alternatively or additionally have an interface for wired communication with an external data processing device, e.g. a USB interface, in particular a USB socket. The interface can optionally also be used to charge an energy storage device, in particular a battery, of the device, for example if the interface is a USB interface.

As shown in FIGS. 1-3, the activating device 120 may include a first connection portion 131, for example a first magnetic element, for attaching the activation device 120 to the aerosol generating device 110. The aerosol generating device 110 may comprise a second connecting part 132, for example a second magnetic element, for coupling to the first connecting part 131. For example, in the coupled state shown in FIG. 3, the activating device 120 and the aerosol generating device 110 are held together due to a magnetic attraction between the first connection part 131 and the second connection part 132.

The activating device 120 can have at least one holding area 135 for holding the activation device with the hand. The activating device 120 illustrated in FIG. 1 has two oppositely arranged holding recesses for engaging one or more fingers in each case.

The aerosol generating device 110 has a nebulizer 111 for generating the aerosol 10, which can be delivered to the nasal cavity of a person via a delivery opening 113. The aerosol generating device 110 further optionally comprises the second control unit 112 for triggering aerosol generation by the nebulizer 111 in response to receiving the activation signal.

FIG. 4 shows the device 100 according to the invention in use in a separated state, in which the activating device 120 and the aerosol generating device 110 are not attached to each other. A person is exhaling into the blow-in (injection) opening 121 until an intra-oral overpressure is detected by means of a pressure sensor 127, which causes a transmitter 125 of the first control unit to send the activation signal 150.

The activation signal 150 is received by the aerosol generating device 110, whereupon the aerosol generation is triggered by the atomizer 111. The aerosol 10 is dispensed into the nose of the person via a nose engagement piece 140 forming the dispensing opening 113. When an activation signal is no longer detected by the aerosol generation device 110, the aerosol generation or aerosol dispensing can be stopped.

The atomizer 111 may use one of the aerosol generation mechanisms described herein. Preferably, the vaporizer 111 comprises a vaporizer that generates the aerosol by vaporizing a substance on a coil. The vaporizer may be constructed in the manner of the electronic vaporizer of an e-cigarette. Alternatively, the atomizer 111 comprises an ultrasonic generator for nebulizing the substance by vibration, in particular in the manner of the ultrasonic nebulizer of an e-cigarette.

The container 117 for the substance can be constructed in the manner of a tank for the liquid of an e-cigarette. The substance can be supplied to the atomizer in a manner analogous to an e-cigarette, namely, for example, passively (for example by means of a capillary effect, such as via a wick) or actively (for example by means of a delivery device, such as a micropump). The design and operation of atomizers of e-cigarettes are known to the skilled person, and reference is made in this context to the relevant literature. However, other types of atomizers are also conceivable, such as the spray atomizers described herein, for example by means of a pressurized gas and/or by means of a nozzle.

FIG. 4 further illustrates that the activating device 120 may optionally include a battery receiving compartment for receiving a first energy storage device 126 in the form of a battery for providing energy to the pressure sensor and/or the first control unit. The aerosol generating device 110 may optionally include a battery receiving compartment for receiving a second energy storage device 116 in the form of a battery for providing energy to the atomizer and/or the second control unit. Accordingly, an easy-to-handle and mobile device with improved functionalities can be provided.

The apparatus 100 may be small, compact and easy to handle. For example, the maximum dimension of the apparatus 100 when coupled (i.e., when the activation device 120 and the aerosol generating device 110 are attached to each other, see FIG. 3) is 20 cm or less, preferably 15 cm or less, more preferably 12 cm or less. The maximum dimension may correspond to a “height H” of the device and is measured along the longitudinal axis L of the device, along which the activation device 120 and the aerosol generation device 110 are arranged side by side in the coupled state. The height of the activating device 120 may be 8 cm or less, preferably 6 cm or less, in particular 4 cm or less. The height of the aerosol generating device 110 can be 12 cm or less, preferably 10 cm or less, in particular 7 cm or less.

A width and/or a depth of the activating device 110 may substantially correspond to a width and/or a depth of the aerosol generating device 120, so that a compact and easy-to-handle device is provided in the coupled state when the activating device 110 is placed on the aerosol generating device 120 in the direction of the longitudinal axis L and coupled thereto (see FIG. 3). For example, the width B of the device is 5 cm or less, in particular 4 cm or less, and/or the depth of the device (measured perpendicular to the paper plane of the drawings) is 3 cm or less, in particular 2 cm or less. A cross-sectional shape of the device (perpendicular to the longitudinal axis L) can be essentially rectangular with rounded corners or oval, e.g. in the manner of a fourth-generation e-cigarette with a “box look”.

The activating device 120 may have a cutout or recess facing towards the aerosol generating device 110, which in the coupled position provides space for an aerosol dispensing channel of the aerosol generating device 110, which may protrude from the atomizer in the direction of the activation device 120. The recess may be shaped substantially complementary to the aerosol dispensing channel, so that the activation device 120 and aerosol generating device 110 substantially complement each other in the coupled state to form a (rounded or angular) “box” (see FIGS. 1-3).

When the mouthpiece 128 is rotated relative to the base body 129 by, for example, 90° to activate the activation device 110, the flat cross-sectional shape of the activating device 110 causes the tubular section formed by the mouthpiece to open downwards. The air exhaled by the person into the mouthpiece can flow through the blowing resistor 123 and be released at the lower end of the mouthpiece past the base body out of the activating device 110 into the ambient air.

The apparatus according to the invention offers reliable triggering of aerosol delivery to the nasal cavity when the soft palate is closed, which is particularly advantageous for medical applications, and is at the same time extremely compact, mobile and easy to use.

The following embodiments are part of the invention:

1. An apparatus (100) for administering an aerosol (10) to the nasal cavity, comprising:
an aerosol generating device (110) having an atomizer (111) for generating the aerosol; and
an activating device (120) comprising:
   a blow-in opening (121);
   a first control unit (122) adapted to transmit an activation signal to the aerosol generating device (110) in response to an exhalation of a person into the blow-in opening (121); and
   a blowing resistor (123), which is designed to resist the person's exhalation.

2. The apparatus according to item 1, wherein the first control unit (122) is adapted to wirelessly activate the aerosol generating device, in particular via a radio or IR signal.

3. The apparatus according to items 1 or 2, wherein the first control unit (122) comprises an integrated circuit (124) and/or a transmitter (125) for transmitting the activation signal to the aerosol generating device (110).

4. The apparatus according to any one of items 1 to 3, wherein the activating device and the aerosol generating device are not attached to each other during use, in particular, are arrangeable physically separate from each other.

5. The apparatus according to one of items 1 to 4, wherein the activating device is arranged to close the soft palate of the person exhaling into the blow-in opening (121).

6. The apparatus according to item 5, wherein the blowing resistor (123) is arranged to generate an intra-oral overpressure in the person exhaling into the blow-in opening (121), which leads to the closure of the person's soft palate.

7. The apparatus according to one of items 1 to 6, wherein the activating device has a mouthpiece (128) forming the blow-in opening, through which the person can exhale into the blow-in opening.

8. The apparatus according to item 7, wherein the mouthpiece (128) is arranged on a base body (129) of the activating device so as to be rotatable about a longitudinal axis (L), in particular wherein a rotation of the mouthpiece (128) by a predetermined angle switches on the activating device.

9. The apparatus according to one of items 1 to 8, wherein the activating device has a pressure sensor (127) which is set up to detect a pressure or a pressure change in the activating device.

10. The apparatus according to one of items 1 to 9, wherein the activating device has an indicator which is set up to generate an indicator signal when a predetermined pressure is reached when the person exhales into the blow-in opening, in particular to generate a visual, acoustic and/or tactile signal.

11. The apparatus according to one of items 1 to 10, wherein the aerosol generating device and the activation device are configured to be detachably attached to one another.

12. The apparatus according to any one of items 1 to 11, wherein the activating device comprises a first connecting part (131) for detachable attaching it to the aerosol generating device.

13. The apparatus according to item 12, wherein the first connecting part (131) comprises at least one first magnetic element which is arranged for coupling to at least one second magnetic element (132) of the aerosol generating device.

14. The apparatus according to one of items 1 to 13, wherein the activating device is arranged to receive a first energy storage device (126), in particular a battery, for providing energy for the activating device, in particular for the first control unit.

15. The apparatus according to any one of items 1 to 14, wherein the activating device comprises at least one holding region (135) for gripping and holding the activating device with a hand or a part thereof.

16. The apparatus according to item 15, wherein the at least one holding region (135) has at least one recess adapted to the engagement of a finger when holding the activation device.

17. The apparatus according to any one of items 1 to 16, wherein the aerosol generating device comprises a second control unit (112) adapted to initiate aerosol generation by the atomizer upon receipt of the activation signal from the activating device.

18. The apparatus according to one of items 1 to 17, wherein the aerosol generating device is arranged to accommodate a second energy storage device (116), in particular a battery, for providing energy for the aerosol generation and/or its triggering.

19. The apparatus according to any one of items 1 to 18, wherein the aerosol generating device further comprises:
a container (117) adapted to contain a substance, wherein the atomizer (111) is adapted to transfer the substance into the aerosol (10); and
a dispensing opening (113) through which the aerosol can be administered to the nasal cavity of the person.

20. The apparatus according to item 19, wherein a first subunit of the aerosol generating device comprising the dispensing opening (113), the atomizer (111) and/or the container (117) is detachably connected to a second subunit of the aerosol generating device comprising a second control unit (112).

21. The apparatus according to items 19 or 20, wherein the aerosol generating device comprises a nose engagement piece (140) forming the dispensing opening.

22. The apparatus according to item 21, wherein the nose engaging piece (140) is arranged on the aerosol generating device so as to be rotatable about an axis of rotation.

23. The apparatus according to any one of items 19 to 22, wherein the dispensing opening (140) is arranged adjacent to the activating device (120) when the activating device and the aerosol generating device are attached to each other.

24. The apparatus according to any one of items 1 to 23, wherein the aerosol generating device comprises an aerosol delivery controller and/or an airflow accelerator for providing a predetermined aerosol flow rate and/or flow velocity.

25. The apparatus according to any one of items 1 to 24, wherein the aerosol generating device comprises at least one heating element for heating the aerosol to a predetermined temperature.

26. The apparatus according to any one of items 1 to 25, wherein the atomizer (111) is adapted to transfer a substance into the aerosol (10) by at least one of the following mechanisms:
heating of the substance via a heating element;
vibration of the substance via an ultrasonic element;
flowing of a compressed gas through the substance;
by means of a vibrating diaphragm;
spraying the substance from a nozzle, in particular using a propellant under pressure; and
by means of an inhalation device.

27. The apparatus according to any one of items 1 to 26, wherein the aerosol generating device comprises a second connecting part for detachably connecting to the activating device, in particular at least one second magnetic element (132) for coupling to at least one first magnetic element (131) of the activating device.

28. The apparatus according to any one of items 1 to 27, wherein the aerosol generating device comprises an indicator adapted to generate an indicator signal indicating an operational state of the aerosol generating device.

29. An activating device (120), in particular of an apparatus (100) according to any one of items 1 to 28, comprising:
a blow-in opening (121);
a first control unit (122) adapted to transmit an activation signal to an aerosol generating device in response to an exhalation of a person into the blow-in opening (121); and
a blowing resistor (123), which is designed to resist the person's exhalation.

30. An aerosol generating device (110), in particular of an apparatus (100) according to any one of items 1 to 28, comprising:
a container (117) for holding a substance;
an atomizer (111) for converting the substance into an aerosol;
a dispensing opening (113) through which the aerosol can be administered to the nasal cavity of a person; and
a second control unit (112) arranged to trigger aerosol generation by the atomizer (111) on receipt of an activation signal from an activating device.

31. Use of a substance in an apparatus according to any of items 1 to 28 to produce an aerosol.

32. Use of an apparatus according to any one of items 1 to 28 for generating an aerosol and for administering the aerosol to the nasal cavity of a person upon exhalation of the person into an blow-in opening of an activating device of the apparatus.

33. A method of generating an aerosol comprising:
exhalation of a person into a blow-in opening of an apparatus for administering an aerosol, in particular an apparatus according to any one of items 1 to 28; and
transmitting an activation signal from the activating device to an aerosol generating device of the apparatus in response to exhalation.

34. A method according to item 33, further comprising:
receiving the activation signal by the aerosol generation device;
triggering aerosol generation by an atomizer in response to receit; and
administering the aerosol to the person's nasal cavity.

While the above description explains preferred embodiments and examples of the invention, the scope of protection is defined by the following claims.

1. An apparatus for administering an aerosol to the nasal cavity, comprising:
an aerosol generating device having an atomizer for generating the aerosol; and
an activating device comprising:
a blow-in opening;
a first control unit adapted to transmit an activation signal to the aerosol generating device in response to an exhalation of a person into the blow-in opening; and
a blowing resistor configured to resist the person's exhalation,
wherein the first control unit is adapted to wirelessly activate the aerosol generating device,
wherein the first control unit comprises an integrated circuit and/or a transmitter for transmitting the activation signal to the aerosol generating device,
wherein the aerosol generating device comprises a second control unit adapted to trigger aerosol generation by the atomizer upon receipt of the activation signal from the activating device,
wherein the aerosol generating device comprises a sensor adapted to determine a position and/or location of the aerosol generating device in relation to the nose or nasal opening of the user,
wherein the sensor is selected from at least one of a distance sensor, a range sensor, and a position sensor, and
wherein the activating device comprises a pressure sensor adapted to detect a pressure or a pressure change in the activating device.

2. (canceled)

3. (canceled)

4. (canceled)

5. (canceled)

6. (canceled)

7. The apparatus according to claim 1, wherein the aerosol generating device comprises an aerosol delivery controller and/or an airflow accelerator; for providing a predetermined aerosol flow rate and/or flow velocity.

8. The apparatus according to claim 1, wherein the aerosol generating device comprises at least one heating element for heating the aerosol to a predetermined temperature for allowing the aerosol to be delivered to the nasal cavity at a predetermined delivery temperature.

9. (canceled)

10. The apparatus according to claim 1, wherein the sensor of the aerosol generating device comprises the position sensor, and the position sensor comprises a magnetometer or gyroscope for determining a position or orientation of the aerosol generating device.

11. (canceled)

12. (canceled)

13. (canceled)

14. (canceled)

15. (canceled)

16. The apparatus according to claim 1, wherein the activating device has a mouthpiece forming the blow-in opening, through which the person can exhale into the blow-in opening.

17. The apparatus according to claim 16, wherein the mouthpiece is arranged on a base body of the activating device so as to be rotatable about a longitudinal axis (L), wherein a rotation of the mouthpiece by a predetermined angle switches on the activating device.

18. The apparatus according to claim 1, wherein the activating device has an indicator which is set up to generate an indicator signal when a predetermined pressure is reached when the person exhales into the blow-in opening, wherein the indicator signal is at least one of a visual, acoustic and tactile signal.

19. The apparatus according to claim 1, wherein the activating device comprises a first connecting part for detachable attaching it to the aerosol generating device.

20. The apparatus according to claim 8, wherein the first connecting part comprises at least one first magnetic element which is arranged for coupling to at least one second magnetic element of the aerosol generating device.

21. The apparatus according to claim 1, wherein the activating device is arranged to receive a first energy storage device for providing energy for the first control unit.

22. The apparatus according to claim 1, wherein the activating device comprises at least one holding region for gripping and holding the activating device with a hand or a part thereof.

23. The apparatus according to claim 22, wherein the at least one holding region has at least one recess adapted to the engagement of a finger when holding the activation device.

24. The apparatus according claim 1, wherein the aerosol generating device further comprises:
a container adapted to contain a substance, wherein the atomizer is adapted to transfer the substance into the aerosol; and
a dispensing opening through which the aerosol can be administered to the nasal cavity of the person,
wherein a first subunit of the aerosol generating device comprising the dispensing opening, the atomizer and/or the container is detachably connected to a second subunit of the aerosol generating device comprising a second control unit.

25. The apparatus according to claim 24, wherein the aerosol generating device comprises a nose engagement piece forming the dispensing opening.

26. The apparatus according to claim 25, wherein the nose engaging piece is arranged on the aerosol generating device so as to be rotatable about an axis of rotation.

27. The apparatus according to claim 1, wherein the first control unit wirelessly activates the aerosol generating device via a radio or IR signal.
